(19) United States
(12) Patent Application Publication
(10) Pub. No.: US 2024/0226341 A9
(43) Pub. Date: Jul. 11, 2024

(54) Dispersion Solution for X-Ray Target, Prefilled Syringe Filled with Same, and Powder for X-Ray Target

(71) Applicant: NATIONAL UNIVERSITY CORPORATION HOKKAIDO UNIVERSITY, Hokkaido (JP)

(72) Inventors: Tetsu Yonezawa, Sapporo-shi, Hokkaido (JP); Hiroki Shirato, Sapporo-shi, Hokkaido (JP); Naoki Miyamoto, Sapporo-shi, Hokkaido (JP)

(73) Assignee: NATIONAL UNIVERSITY CORPORATION HOKKAIDO UNIVERSITY, Hokkaido (JP)

(21) Appl. No.: 18/278,863

(22) PCT Filed: Feb. 25, 2022
(86) PCT No.: PCT/JP2022/008039

§ 371 Date: Aug. 25, 2023

(30) Foreign Application Priority Data
Feb. 26, 2021 (JP) 2021-029450

(51) Int. Cl. A61K 49/04

(52) U.S. Cl. CPC A61K 49/0428

(57) ABSTRACT

A dispersion for an X-ray target in which gold nanoparticles and sodium alginate or a calcium phosphate-based bone reinforcing material are dispersed, in which the gold nanoparticles are in contact with the sodium alginate or the calcium phosphate-based bone reinforcing material.

### TECHNICAL FIELD

The present disclosure relates to a dispersion for an X-ray target, a prefilled syringe filled with same, and a powder for an X-ray target.

### BACKGROUND ART

In radiation therapy, high-precision X-ray therapy has a good dose distribution, and a certain effect has been achieved by combination use of a molecular targeting agent and/or an anticancer agent. However, tumors such as lung cancer and liver cancer move and change their position each time of breathing. For example, in lung cancer near the diaphragm, the position of the tumor moves with an amplitude of about 3 cm. At this time, for example, when a tumor having a diameter of 1 cm moves laterally with an amplitude of 1.5 cm, it is necessary to irradiate a 4 cm area including the tumor with radiation at the time of radiation therapy. When the irradiation range is widened, the normal site is also irradiated with radiation. Therefore, the radiation dose needs to be reduced so that the normal tissue can tolerate the radiation dose, which extremely reduces the therapeutic effect and damages many normal cells.

In order to solve the above problem, it is effective if a radiopaque lesion identification target (hereinafter, also referred to as an “X-ray target”) is introduced into the body and the target can be irradiated. iGold (registered trademark) (manufactured by MEDIKIT CO., LTD.) has been mainly used as a target so far. This is composed of gold particles having a diameter of about 1.5 mm or 2.0 mm, and is introduced into the body with a puncture kit having a diameter of 2.55 mmφ.

In addition, Patent Document 1 discloses a lesion identification marker for radiation therapy, which is, for example, a mixture of a calcium phosphate-based bone reinforcing material and gold particles.

### CONVENTIONAL ART DOCUMENT

### Patent Document

Patent Document 1: WO 2018/038223 A

### DISCLOSURE OF THE INVENTION

### Problems to be Solved by the Invention

However, introducing iGold (registered trademark) into the body with a 2.55 mmφ puncture kit is highly invasive. In addition, although the introduction of the lesion identification marker disclosed in Patent Document 1 into the body is less invasive than the introduction of iGold (registered trademark) into the body, it is necessary to mix the calcium phosphate-based bone reinforcing material and the gold particles immediately before the introduction into the body, and it is necessary to adjust the particle size by sieving the gold particles, and the like. Thus, a heavy burden is on the operator and the like.

The present disclosure has been made in view of such a situation, and an object thereof is to provide a dispersion for an X-ray target, a prefilled syringe filled with the dispersion, and a powder for an X-ray target, in which sufficient X-ray visibility can be obtained, and at the time of introducing an X-ray target into a body, the dispersion for an X-ray target is less invasive and a burden on an operator or the like can be reduced as compared with a case of introduction with a conventional puncture kit.

### Means for Solving the Problems

The present invention according to a first aspect provides a dispersion for an X-ray target in which gold nanoparticles and sodium alginate or a calcium phosphate-based bone reinforcing material are dispersed,
in which the gold nanoparticles are in contact with the sodium alginate or the calcium phosphate-based bone reinforcing material.

The present invention according to a second aspect provides a prefilled syringe filled with the dispersion according to the first aspect in which the gold nanoparticles and the sodium alginate are dispersed and a calcium ion solution in a non-mixed state.

The present invention according to a third aspect provides the dispersion according to the first aspect, in which the gold nanoparticles and the sodium alginate are dispersed, and calcium carbonate and lactones are further contained.

The present invention according to a fourth aspect provides a prefilled syringe filled with the dispersion according to the third aspect.

The present invention according to a fifth aspect provides a powder for an X-ray target including a gold nanoparticle and a calcium phosphate-based bone reinforcing material,
in which the gold nanoparticle is in contact with the calcium phosphate-based bone reinforcing material.

### Effects of the Invention

According to embodiments of the present invention, it is possible to provide a dispersion for an X-ray target, a prefilled syringe filled with the dispersion, and a powder for an X-ray target, in which sufficient X-ray visibility can be obtained, and at the time of introducing an X-ray target into a body, the dispersion for an X-ray target is less invasive and a burden on an operator or the like can be reduced as compared with a case of introduction with a conventional puncture kit.

### BRIEF DESCRIPTION OF THE DRAWINGS

FIG. 1 shows a result of determining a particle size distribution of a dispersion of Example 1 by TEM observation when a plasma reaction time is changed from 10 to 25 minutes.

FIG. 2 shows a photograph after injecting the dispersion of Example 1 into a simulated organ gel and subsequently injecting an aqueous calcium chloride solution.

FIG. 3 shows a photograph (left figure) and a cross-sectional SEM image (right figure) of an X-ray target obtained by aggregating the dispersion of Example 1 in a simulated organ gel.

FIG. 4 shows photographs of X-ray targets (Sample Nos. 1-1 to 1-7) obtained by aggregating the dispersion of Example 1 in simulated organ gels.

FIG. 5 shows X-ray fluoroscopic images of Sample Nos. 1-1 to 1-7.

FIG. 6 shows photographs of a dispersion of Example 2 immediately after the temperature was adjusted to each temperature (left figure) and after the dispersion was left at each temperature for 1 day (right figure).

FIG. 7 shows a result of viscoelasticity measurement when the temperature of the dispersion of Example 2 was changed from 4° C. to 37° C.

FIG. 8 shows a result of examining a force applied when the dispersion of Example 2 was injected with an injection needle.

FIG. 9 shows X-ray fluoroscopic images of Sample Nos. 2-1 to 2-7.

FIG. 10 is TEM images of powders of Example 3.

FIG. 11 shows a photograph of an X-ray target in which a dispersion of Example 3 was aggregated in a simulated organ gel.

FIG. 12 is a schematic view for explaining an in-liquid plasma method performed in Example 4.

FIG. 13A is an example of a TEM image before centrifugation of gold nanoparticles whose surface is covered with sodium alginate in Example 4.

FIG. 13B is an example of a TEM image after centrifugation of gold nanoparticles whose surface is covered with sodium alginate in Example 4.

FIG. 13C shows a particle size distribution of gold nanoparticles determined from a region of a TEM image after centrifugation in Example 4.

FIG. 14 shows a photograph after injecting a dispersion of Example 4 into a simulated organ gel, and subsequently injecting an aqueous solution of calcium chloride and magnesium chloride hexahydrate.

FIG. 15A shows a surface image of an X-ray target obtained by aggregating the dispersion of Example 4 in the simulated organ gel, photographed with a digital microscope.

FIG. 15B shows a surface SEM image of an X-ray target obtained by aggregating the dispersion of Example 4 in the simulated organ gel.

### MODE FOR CARRYING OUT THE INVENTION

The present inventors have studied from various angles in order to realize a dispersion for an X-ray target, in which sufficient X-ray visibility can be obtained, and at the time of introducing an X-ray target into a body, the dispersion for an X-ray target is less invasive and a burden on an operator or the like can be reduced as compared with a case of introduction with a conventional puncture kit.

In order to make it less invasive, it is conceivable to make the gold particles small. However, if the gold particles are simply made small, visibility as an X-ray target will be insufficient. In addition, as disclosed in Patent Document 1, if the gold particles are too small, aggregation between the gold particles is occurred. Particularly, in a product (manufactured by The Nilaco Corporation) having a particle size of 1 to 2 μm, a particle mass of several hundreds μm or more may be included.

The present inventors have found a dispersion for an X-ray target in which a biocompatible material that can be aggregated by a chemical reaction (for example, sodium alginate or calcium phosphate-based bone reinforcing material) and gold nanoparticles in contact with these materials are dispersed. It has been found that: the dispersion can suppress aggregation between gold nanoparticles before introduction into the body; the dispersion can be introduced with an injection needle or a catheter at the time of introduction into the body (that is, less invasive than introduction with a conventional puncture kit); and sufficient visibility as an X-ray target can be obtained by aggregation by a chemical reaction after introduction into the body. Furthermore, it has been found that: by using the dispersion, it is not necessary for the operator or the like to mix a material such as a calcium phosphate-based bone reinforcing material and gold particles; it is not necessary to adjust the particle size by sieving or the like the gold particles; and the burden on the operator or the like can be reduced.

Hereinafter, details of each requirement defined by the embodiments of the present invention will be described.

<Dispersion for X-ray Target>

A dispersion for an X-ray target according to embodiments of the present invention is a dispersion for an X-ray target in which a material which is biocompatible and can be aggregated by a chemical reaction (hereinafter, may be simply referred to as an “aggregatable material”) and gold nanoparticles with which the material is in contact are dispersed.

Here, the term “biocompatible” refers to a property of having affinity with biological tissues and organs and not causing a foreign body reaction, a rejection reaction, and the like. Specific examples of the biocompatible material include known food additives, known medicines, known biomaterials (materials for artificial joint, dental implant, artificial bone and artificial blood vessel, etc.), and the like. Owing to the biocompatibility, it can be used as materials for X-ray targets (that is, for introduction into the body).

The material that can be aggregated by a chemical reaction may be any material as long as it can form an aggregate with a chemical change. For example, it is preferable that an aggregate having a size equal to or larger than that of conventional iGold (registered trademark) (that is, 1.5 mmφ or more) can be formed. As a result, by causing a predetermined chemical change after introduction into the body, an aggregate containing gold is formed, and sufficient X-ray visibility is easily ensured.

The aggregatable material may be water soluble or water-insoluble.

If the aggregatable material is water-soluble, for example, when the dispersion medium is water, it enables that: gold nanoparticles whose surface is covered with the aggregatable material is obtained; the particles in the dispersion are made smaller; the particles are dispersed separately; and even if the diameter of an injection needle for introducing the dispersion into the body is reduced, the particles easily pass through the injection needle to be made less invasive.

If the aggregatable material is water-insoluble, for example, when the dispersion medium is water, it enables that: a dispersion with the aggregatable material having a large particle size to some extent (for example, more than 2 μm in average particle size (median diameter)) in contact with the surface of the gold nanoparticles is obtained; and for example, as described later, the aggregatable material is also used as a powder for an X-ray target with the aggregatable material in contact with the surface of the gold nanoparticles. On the other hand, since the particle size of the aggregatable material is small (for example, 15 μm or less in terms of an average particle size (median diameter)), even if the diameter of the injection needle for introducing the dispersion into the body (although not as small as that in the case where the aggregatable material is water-soluble) is reduced, it becomes easy to pass through the injection needle.

An example of an aggregatable material is sodium alginate. Sodium alginate is water-soluble and can undergo the following chemical reaction with calcium ions (Ca²⁺) to form an aggregate.

2NaAlg+Ca²⁺→Ca(Alg)₂+2Na⁺ (1)

In the above formula, “Alg” refers to alginic acid (C₆H₇O₆).

As sodium alginate, one having a high viscosity is preferable. By using sodium alginate having a high viscosity, the gold nanoparticles are in good contact with the sodium alginate (that is, the gold nanoparticle surface is well covered), and the gold nanoparticles can be dispersed in a dispersion medium such as water at a high concentration. Specifically, sodium alginate having a viscosity of 800 mPa·s or more in a 1% aqueous solution at 20° C. is preferable, and examples thereof include 1-8 manufactured by KIMICA Corporation.

Other examples of the aggregatable materials include a calcium phosphate-based bone reinforcing material (also referred to as calcium phosphate cement). The calcium phosphate-based bone reinforcing material is a water-insoluble calcium phosphate-based composition. For example, the calcium phosphate-based bone reinforcing material described in JP-2002-255603 A, JP-2002-291866 A, JP-564-037445 A, and JP-2010-075247 A is known. It is preferable that the calcium phosphate-based bone reinforcing material contains at least one selected from the group consisting of α-tricalcium phosphate, tetracalcium phosphate, calcium hydrogen phosphate, and β-tricalcium phosphate. These can be converted to hydroxyapatite (Ca₁₀(PO₄)₆(OH)₂) by a chemical reaction (hydration reaction) to form an aggregate. For example, tricalcium phosphate (Ca₃(PO₄)₂) can cause the following chemical reaction to form an aggregate.

10Ca₃(PO₄)₂+6H₂O→3Ca₁₀(PO₄)₆(OH)₂+3H⁺+PO₄³⁻ (2)

Examples of commercially available calcium phosphate-based bone reinforcing materials include: BIOPEX (registered trademark)-R (standard type, long type, or excellent type) (manufactured by HOYA Technosurgical Co., Ltd.) containing α-type tricalcium phosphate (75 mass %), tetracalcium phosphate (18 mass %), calcium hydrogen phosphate (5 mass %), hydroxyapatite (2 mass %) and magnesium phosphate; and Cerapaste (manufactured by Niterra Co., Ltd.) which is a mixed composition of tetracalcium phosphate and anhydrous calcium hydrogen phosphate; and the like.

In the dispersion for an X-ray target according to the embodiments of the present invention, gold nanoparticles are dispersed in addition to the aggregatable material. Gold is biocompatible and has good X-ray visibility. The average particle size (median diameter) of the gold nanoparticles is preferably less than 1 μm, more preferably 500 nm or less, and still more preferably 100 nm or less.

By using the gold nanoparticles, even if the diameter of the injection needle to be introduced into the body is reduced, the dispersion can easily pass the injection needle. That is, the dispersion can be made less invasive. When the aggregatable material is water-soluble and the dispersion medium is water, for example, the dispersion can pass an injection needle of 25 G (inner diameter: about 0.25 mm) or thinner by using gold nanoparticles. When the aggregatable material is water-insoluble and the dispersion medium is water, for example, the dispersion can pass an injection needle of 21 G (inner diameter: about 0.59 mm) or thinner by using gold nanoparticles.

In the dispersion for an X-ray target according to the embodiments of the present invention, the gold nanoparticles are in contact with the aggregatable material. This makes it possible to suppress aggregation of the gold nanoparticles before introduction into the body. As a method for confirming whether the gold nanoparticles are in contact with the aggregatable material, a known method can be used. For example, the gold nanoparticles can be observed with a transmission electron microscope (TEM). Alternatively, gold nanoparticles covered with sodium alginate can be confirmed by a known mass spectrometry. For example, sodium alginate on the surface is ionized by laser desorption ionization (LDI) or the like, and the mass-to-charge ratio is determined by time of flight (TOF) or the like, whereby it can be confirmed that the gold nanoparticles and sodium alginate are in contact with each other (that is, the surface of the gold nanoparticles is covered with sodium alginate).

As a method for bringing the aggregatable material and the gold nanoparticles into contact with each other, merely separately preparing and bringing the aggregatable material and the gold nanoparticles into contact with each other may cause aggregation between the gold nanoparticles. Therefore, examples of the method for contacting include a method in which chloroauric acid is reduced by an in-liquid plasma method or an alcohol reduction method in the presence of an aggregatable material. When the aggregatable material is water-insoluble, the dispersibility can be improved by the in-liquid plasma method, and the alcohol reduction method is preferable for suppressing the modification of the aggregatable material. When the aggregatable material is water-soluble, it is preferable that the solvent is only water by the in-liquid plasma method because the solubility of the aggregatable material can be increased and the contact efficiency can be increased. Other examples include a method in which the gold rod is made into nanoparticles by an in-liquid plasma method in the presence of an aggregatable material. By these methods, a dispersion in which the aggregatable material and the gold nanoparticles are in contact with each other is obtained, and aggregation between the gold nanoparticles can be suppressed.

The dispersion medium of the dispersion for an X-ray target according to the embodiments of the present invention is not particularly limited, and water such as water for injection is preferable from the viewpoint of biocompatibility.

With respect to the mixing ratio in the dispersion, for example, when the mixing ratio of the aggregatable material and/or the gold nanoparticles is large, an aggregate can be formed with a small introduction amount of the dispersion and/or X-ray visibility can be ensured. On the other hand, when the amount of the dispersion medium is large, fluidity of the liquid can be ensured. Therefore, the mixing ratio may be appropriately adjusted according to the characteristics and the like of the aggregatable material and the dispersion medium. The dispersion for an X-ray target according to the embodiments of the present invention may contain other materials within the range of achieving the object of the present invention.

The dispersion for an X-ray target according to the embodiments of the present invention is introduced into a body by an injection needle, a catheter, or the like, and then causes a predetermined chemical reaction according to an aggregatable material to form an aggregate, so that an X-ray target can be obtained. The predetermined chemical reaction can be caused (or promoted) after introduction into the body by mixing with a substance (including ions) necessary for the reaction, a change with time, a change in temperature, and the like.

As the X-ray target, the more gold is contained, the higher the X-ray visibility is. For example, it is preferable that 1.5 mg or more of gold is contained, more preferably 10.0 mg or more of gold is contained, and still more preferably 22.0 mg or more of gold is contained. It is preferable to adjust the gold concentration in the dispersion and the introduction amount of the dispersion into the body so as to have such a gold content.

A specific example of the method for using the dispersion for an X-ray target according to the embodiments of the present invention will be described.

As described above, an example of the aggregatable material includes sodium alginate.

By covering the surfaces of the gold nanoparticles with sodium alginate having a high viscosity, the gold nanoparticles can be dispersed in a dispersion medium such as water at a high concentration to some extent, and for example, the gold nanoparticle concentration can be set to 0.04 to 2.0 mg/mm³. The concentration of sodium alginate in the dispersion is not particularly limited, but may be, for example, 0.1 to 20.0% (mass/volume (g/ml) ratio), and the mixing ratio of gold nanoparticles:sodium alginate is not particularly limited, and may be, for example, 1:0.01 to 1:100 in a weight ratio.

In the above formula (1), since sodium alginate and calcium ions (Ca 2+) have high reactivity, when Ca²⁺ is contained in the dispersion, a chemical reaction of the above formula (1) occurs before introduction into the body. When the reactivity is high as described above, it is conceivable to introduce a liquid containing a substance necessary for the reaction (in this case, a Ca²⁺ solution) into the body after introducing a dispersion containing an aggregatable material into the body. Alternatively, for example, calcium carbonate that releases Ca²⁺ under acidity and lactones that hydrolyze with an increase in temperature to generate a carboxylic acid may be contained in the dispersion containing the aggregatable material. In this case, by introducing the dispersion into the body, the lactones can generate a carboxylic acid, and the calcium carbonate can release Ca²⁺ to cause a chemical reaction of the formula (1). Examples of such lactones include gluconodeltalactone, lactic acid lactone, glycolic acid lactone, and D-pantolactone, and it is preferable to contain one or more of these lactones. The concentration of the lactones in the dispersion is not particularly limited, and may be, for example, 0.01% or more and 1.00% or less (mass/volume (g/ml) ratio). The concentration of calcium carbonate may be, for example, 0.01% or more and 1.00% or less (mass/volume (g/ml) ratio) based on the amount of calcium ions.

In addition, as an example of the aggregatable material, a calcium phosphate-based bone reinforcing material is also exemplified. In the dispersion (or in the powder), the mixing ratio of the gold nanoparticles:the calcium phosphate-based bone reinforcing material is preferably 1:0.5 to 1:4 in a weight ratio. In addition, by bringing the gold nanoparticles into contact with (carrying) the calcium phosphate-based bone reinforcing material, the gold nanoparticles can be dispersed at a high concentration to some extent in the dispersion. For example, the concentration of the gold nanoparticles can be set to 0.04 to 2.0 mg/mm³. The concentration of the calcium phosphate-based bone reinforcing material is not particularly limited, and can be set to 2 to 500% (mass/volume (g/ml) ratio).

The reactivity of the hydration reaction as in the above formula (2) is not so high, and it is preferable to mix the dispersion with the reaction promoting liquid immediately before introducing the dispersion into the body. Examples of the reaction promoting liquid include: a liquid including one or more kinds of readily water-soluble halides, sulfates and organic acid salts, water and an acid (for example, hydrochloric acid, sulfuric acid, phosphoric acid, formic acid, acetic acid, succinic acid, lactic acid, and the like) (refer to JP-S59-88351 A); a liquid in which an acidic solution containing a monomer or a copolymer of an unsaturated carboxylic acid (for example, acrylic acid, maleic acid, fumaric acid, itaconic acid) is used (refer to JP-S60-253454 A); a liquid containing an antibacterial agent (for example, propylene glycol, ethylene glycol, and the like) and a water-soluble polymer (for example, chitin, chitosan, soluble starch, chondroitin sulfate and salts thereof, carboxymethyl cellulose, and the like) (refer to JP-H3-267067 A); and a liquid containing a water-soluble sodium salt such as sodium succinate (refer to JP-H4-12044 A). The following liquids may be preferably used that: a liquid containing sodium chondroitin sulfate (sodium chondroitin sulfate) in combination with the above, disodium succinate anhydride, sodium bisulfite and water such as water for injection (Japanese Pharmacopoeia), etc. (refer to JP-2002-255603 A); a liquid containing dextran sulfate sodium sulfur 5 (dextran sulfate sodium sulfur 5) and water such as water for injection (refer to JP-2002-291866 A), etc.; a kneading liquid consisting of water such as water for injection; a liquid containing water-soluble sodium salts such as sodium phosphate; and a kneading liquid containing various organic acids such as citric acid, or the like. As the reaction promoting liquid, for example, a dedicated kneading liquid of BIOPEX (registered trademark)-R (manufactured by HOYA Technosurgical) containing disodium succinate anhydride (12 mass %), sodium chondroitin sulfate (5 mass %), sodium bisulfite, and water for injection (83 mass %), a cured liquid of Cerapaste (Composition: dextran sulfate sodium sulfur 5, water for injection), and the like can be used.

In the dispersion, the volume of the reaction promoting liquid per 1 g of the calcium phosphate-based bone reinforcing material is preferably 0.1 mL/g to 0.5 mL/g.

By mixing with the reaction promoting liquid as described above and then introducing into the body with an injection needle or the like, the hydration reaction as in the above formula (2) proceeds after introduction into the body to form an aggregate.

<Prefilled Syringe>

A prefilled syringe according to the embodiments of the present invention is a syringe filled with the dispersion. By using such a syringe, it is not necessary to fill the syringe with the dispersion after measuring the dispersion, and the dispersion can be immediately introduced into the body, so that the burden on the operator or the like can be further reduced.

In the case of high reactivity such as sodium alginate and Ca′, if the dispersion for an X-ray target and a liquid containing a substance necessary for the reaction (in this case, a Ca²⁺ solution) are filled together (in a state where they can be mixed), the reaction may proceed before injection, and therefore it is preferable to separately fill these liquids (in a non-mixed state). For example, one container may be filled with a dispersion for an X-ray target and the other container may be filled with a Ca²⁺ solution using a prefilled syringe (for example, a two-liquid mixed administration device (manufactured by NIPRO CORPORATION) or the like) in which two containers are arranged in parallel and configured such that the liquids in the two containers can be mixed at the same time as the injection. Alternatively, using a two-chamber prefilled syringe (also referred to as a double chamber syringe) in which the front end side and the rear end side of one container are partitioned by a partition wall, the dispersion for an X-ray target may be filled on the front end side, the Ca²⁺ solution may be filled on the rear end side, and the Ca²⁺ solution may be injected after the dispersion for an X-ray target is injected. Examples of the calcium ion (CO solution include an aqueous calcium chloride solution.

In addition, in the case of a dispersion (for example, the aggregatable material is sodium alginate, and the dispersion for an X-ray target further contains calcium carbonate and lactones) which does not react (aggregate) at a low temperature, it is preferable that the dispersion is filled in a one-chamber type prefilled syringe (also referred to as a single-chamber syringe) and stored at a low temperature.

In the dispersion filled in the prefilled syringe according to the embodiments of the present invention, the more gold is contained, the higher the X-ray visibility as an X-ray target is. For example, it is preferable that 1.5 mg or more, more preferably 10.0 mg or more, and still more preferably 22.0 mg or more of gold is contained.

<Powder for X-Ray Target>

In the dispersion for an X-ray target according to the embodiments of the present invention, when the aggregatable material has low solubility in the dispersion medium and the particle size of the aggregatable material is large to some extent (for example, more than 2 μm in average particle size (median diameter)), only the particles in the dispersion may be taken out to obtain a powder for an X-ray target. The powder includes a biocompatible material that can be aggregated by a chemical reaction, and gold nanoparticles in contact with the material. By using the powder, aggregation between the particles can be suppressed before introduction into the body, the operator or the like does not need to mix the aggregatable material and the gold particles, and the gold particles do not need to be sieved or the like to adjust the particle size. By dispersing the powder in an appropriate dispersion medium (water for injection or the like), the dispersion can be introduced into the body by an injection needle, a catheter or the like. After the dispersion is introduced into the body, the sufficient visibility as an X-ray target can be ensured by aggregating it with a chemical reaction.

An example of the powder for an X-ray target according to the embodiments of the present invention includes a powder containing gold nanoparticles and a calcium phosphate-based bone reinforcing material, in which the gold nanoparticles are in contact with the calcium phosphate-based bone reinforcing material (the gold nanoparticles are supported on the calcium phosphate-based bone reinforcing material). By dispersing the powder in a dispersion medium such as water for injection, the dispersion for an X-ray target according to the embodiment of the present invention can be easily obtained.

### EXAMPLES

The embodiments of the present invention will be described in more detail by way of Examples. It is to be understood that the embodiments of the present invention are not limited to the following Examples, and various design variations made in accordance with the purports mentioned hereinbefore and hereinafter are also included in the scope of the embodiments of the present invention.

### Example 1

Chloroauric acid was reduced by an in-liquid plasma method in the presence of sodium alginate as an aggregatable material to obtain a dispersion of gold nanoparticles whose surface was covered with sodium alginate.

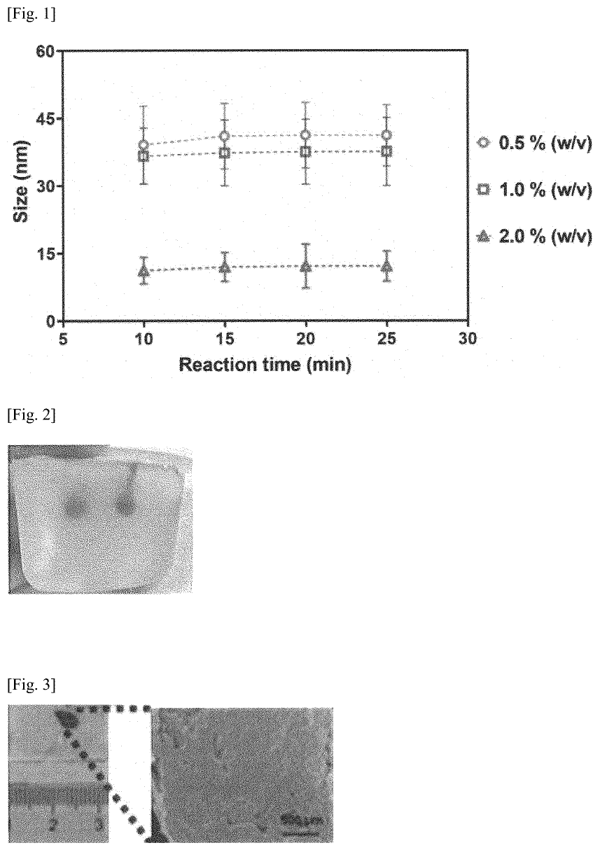

Specifically, sodium alginate (I-8, manufactured by Kimica Corporation) was dissolved in a proportion of 0.5, 1.0 or 2.0 g with respect to 100 mL of pure water to prepare 0.5, 1.0 or 2.0% (weight/volume (g/ml) ratio) sodium alginate aqueous solution. On the other hand, 4.08 mL of a 24.6 mM chloroauric acid aqueous solution was dissolved in 195.92 mL of pure water to prepare a 0.5 mM chloroauric acid solution. The two solutions were mixed in a ratio of alginic acid aqueous solution:chloroauric acid aqueous solution being 1:2. This mixed solution was stirred for 3 hours and introduced into the plasma reaction solution. Plasma was generated with microwave energy of 500 W to obtain a dispersion of gold nanoparticles whose surface was covered with sodium alginate. In addition, samples were produced by changing the plasma reaction time from 10 to 25 minutes, and the particle size distributions were determined by observation with a transmission electron microscope (TEM). The results are illustrated in FIG. 1. In FIG. 1, the horizontal axis represents the plasma reaction time (minutes), and the vertical axis represents the particle size (nm). As shown in FIG. 1, the particle size ranges of the samples using 0.5% (indicated by a circle) and 1.0% (indicated by a square) of an aqueous sodium alginate solution were 30 to 50 nm (central value was 35 to 40 nm), and the particle size range of the sample using 2.0% (indicated by a triangle) of an aqueous sodium alginate solution was 5 to 20 nm (central value was 10 to 15 nm). It is considered that the average particle size (median diameter) was 50 nm or less at most under any conditions. The obtained gold nanoparticles were stably dispersed in water, and this is because sodium alginate was in contact with and covered the surfaces of the gold nanoparticles, enabling them to be dispersed in water.

An aggregation experiment in a simulated organ gel was performed using a dispersion for an X-ray target (gold nanoparticle concentration: 0.5 mg/mm³, sodium alginate concentration: 2.5% (mass/volume (g/ml) ratio)) prepared by redispersing the gold nanoparticles (obtained by using 0.5% sodium alginate aqueous solution) whose surface was covered with sodium alginate obtained as described above in water. FIG. 2 shows photographs after injecting the dispersion into two places in the simulated organ gel (KONNYAKUBATAKE, manufactured by MannanLife CO., LTD.) with an injection needle (21 G (inner diameter: about 0.59 mm, needle length: about 38.1 mm)) and subsequently injecting an aqueous calcium chloride solution. As shown in FIG. 2, by using the dispersion, aggregates (dark color portions) could be formed at two places in the simulated organ gel.

FIG. 3 shows a photograph (left figure) and a cross-sectional SEM image (right figure) of the X-ray target aggregated in the simulated organ gel as described above. As can be seen from the SEM image, the aggregate (X-ray target) obtained as described above has no (few) voids and forms a dense solid.

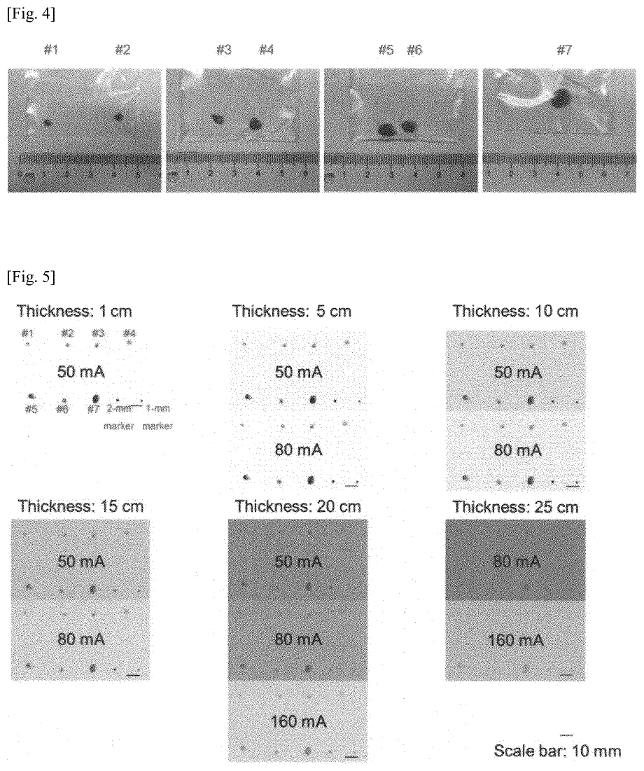

Using the dispersion for an X-ray target obtained as described above, aggregates (X-ray target) were produced in a simulated organ gel, and X-ray visibility was examined. The size of the X-ray target was changed in 7 stages (Sample Nos. 1-1 to 1-7), and photographs of Sample Nos. 1-1 to 1-7 (denoted as #1 to #7 in FIG. 4) actually produced are shown in FIG. 4, and summarized sample schematic dimensions, sample volumes, gold nanoparticle concentrations, and gold nanoparticle amounts are shown in Table 1. As a method for examining X-ray visibility, Sample Nos. 1-1 to 1-7 were arranged on a 96 hole plate, placed on an acrylic phantom, and an X-ray fluoroscopic images were obtained by an X-ray fluoroscope (X-ray generator: UD 150B-40 manufactured by Shimadzu Corporation, bulb: tungsten, flat panel detector for obtaining X-ray images: PaxScan 3030 manufactured by Varian Medical Systems, Inc.). As a positive control, pure gold spherical markers (iGold (registered trademark)) with diameters of 1.5 mm and 2.0 mm currently in clinical use were placed. The thickness of the acrylic plate was changed stepwisely from 1 cm to 25 cm. The tube voltage of the X-ray generator was fixed at 110 kV. The exposure time was fixed at 3 msec. The tube current was selected from 50 mA, 80 mA, and 160 mA depending on the situation. About 100 X-ray fluoroscopic images were obtained under each condition. Under each condition, an image of an X-ray target to be evaluated was cut out from one of the plurality of images to create a template image. Template pattern matching was performed on the other images by normalized cross-correlation with the template image created in advance. When an average value of correlation coefficients (%) obtained from the template pattern matching on about 100 images exceeded 30%, it was determined that image recognition was possible, and when the average value was less than or equal to 30%, it was determined that image recognition was impossible. An image processing library (Matrox Imaging Library 9 manufactured by Matrox Corporation) was used for gradation treatment and pattern matching of an image. FIG. 5 shows the X-ray fluoroscopic images of Sample Nos. 1-1 to 1-7, and Table 2 shows the visibility evaluation results (average values (%) of correlation coefficients). Note that, in FIG. 5, those denoted as #1 to #7 are fluoroscopic images of Sample Nos. 1-1 to 1-7, respectively, and those denoted as “2-mm marker” and “1-mm marker” are fluoroscopic images of pure gold spherical markers having diameters of 2.0 mm and 1.5 mm, respectively, and an acrylic plate thickness (thickness) and a tube current (a numerical value with an ending of “mA”) are added.

TABLE 1

| | | | Gold | Gold |
|---|---|---|---|---|
| | Sample schematic | Sample | nanoparticle | nanoparticle |
| Sample | dimensions | volume | concentration | amount |
| Nos. | (mm × mm × mm) | (mm³) | (mg/mm³) | (mg) |
| 1-1 | 2.0 × 2.0 × 3.0 | 6.28 | 0.5 | 3.14 |
| 1-2 | 2.0 × 2.5 × 3.0 | 7.85 | 0.5 | 3.93 |
| 1-3 | 3.0 × 3.0 × 5.0 | 23.56 | 0.5 | 11.78 |
| 1-4 | 4.0 × 4.0 × 5.0 | 41.89 | 0.5 | 20.95 |
| 1-5 | 3.0 × 5.0 × 6.0 | 62.83 | 0.5 | 31.42 |
| 1-6 | 4.0 × 4.0 × 5.0 | 41.89 | 0.5 | 20.95 |
| 1-7 | 5.0 × 6.0 × 8.0 | 125.66 | 0.5 | 62.83 |

TABLE 2

| | Acrylic | Sample Nos. | | | | | | | iGold (registered trademark) | |
|---|---|---|---|---|---|---|---|---|---|---|
| Tube | plate | 1-1 | 1-2 | 1-3 | 1-4 | 1-5 | 1-6 | 1-7 | 1.5 mmφ | 2.0 mmφ |
| current | thickness | Gold | Gold | Gold | Gold | Gold | Gold | Gold | Gold | Gold |
| (mA) | (cm) | 3.14 mg | 3.93 mg | 11.78 mg | 20.95 mg | 31.42 mg | 20.95 mg | 62.83 mg | 34.2 mg | 80.95 mg |
| 50 | 1 | 98.27 | 98.51 | 99.06 | 98.16 | 99.55 | 98.98 | 100.00 | 99.12 | 99.5 |
| 50 | 5 | 93.48 | 95.36 | 97.26 | 95.29 | 98.31 | 96.94 | 100.00 | 97.58 | 98.85 |
| 80 | 5 | 96.50 | 97.46 | 98.48 | 96.97 | 98.96 | 98.32 | 100.00 | 98.60 | 99.26 |
| 50 | 10 | 80.97 | 82.04 | 89.17 | 78.93 | 92.47 | 87.84 | 97.65 | 90.54 | 94.62 |
| 80 | 10 | 88.76 | 89.75 | 93.3 | 88.46 | 96.33 | 93.20 | 100.00 | 95.25 | 97.27 |
| 50 | 15 | 42.89 | 49.17 | 61.63 | 43.76 | 75.74 | 60.28 | 83.34 | 66.66 | 81.32 |
| 80 | 15 | 61.70 | 66.79 | 74.31 | 63.44 | 84.75 | 72.75 | 92.49 | 80.27 | 89.27 |
| 50 | 20 | 2.40 | 1.41 | 1.73 | 1.00 | 32.79 | 2.08 | 39.23 | 25.20 | 41.62 |
| 80 | 20 | 16.75 | 24.95 | 31.49 | 4.47 | 50.76 | 31.22 | 58.79 | 43.21 | 57.01 |
| 160 | 20 | 37.60 | 39.80 | 51.84 | 29.25 | 70.45 | 46.57 | 79.57 | 63.51 | 72.60 |
| 80 | 25 | 1.00 | 1.00 | 1.00 | 1.00 | 1.23 | 1.00 | 1.00 | 1.21 | 4.81 |
| 160 | 25 | 1.46 | 1.21 | 2.77 | 1.00 | 10.10 | 1.00 | 27.92 | 1.69 | 28.51 |

As can be seen from Table 2, in Sample Nos. 1-1 to 1-7, the gold contents were 1.5 mg or more, and sufficient X-ray visibilities (specifically, the average value of correlation coefficients exceeds 30% at a tube current of 50 mA and an acrylic plate of 10 cm) were ensured. Further, in particular, in Sample No. 1-5 (gold content: 31.42 mg) and Sample No. 1-7 (gold content: 62.83 mg) having a gold content of 22.0 mg or more, X-ray visibilities (average value (%) of correlation coefficient) substantially equivalent to that of iGold (registered trademark) were ensured.

### Example 2

A dispersion for an X-ray target (gold nanoparticle concentration: 0.5 mg/mm³, sodium alginate concentration: 2.5% (mass/volume (g/ml) ratio)), GDL concentration: 0.15% (mass/volume (g/ml) ratio), and calcium ion concentration: 0.075% (mass/volume (g/ml) ratio) was produced by adding gluconodeltalactone (GDL) and calcium carbonate into a dispersion of gold nanoparticles whose surface was covered with sodium alginate prepared in the same manner as in Example 1.

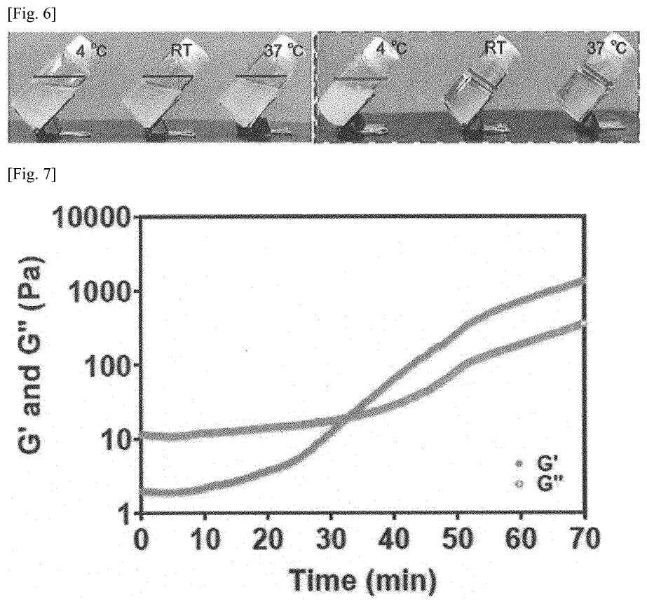

FIG. 6 shows photographs of the dispersion immediately after the temperature was adjusted to each temperature (left figure) and after the dispersion was left at each temperature for 1 day (right figure). In FIG. 6, a straight line parallel to the liquid level is added to facilitate discrimination of the liquid level. It can be found that the dispersion did not gelate at 4° C., but gelated after being left for 1 day at room temperature (RT) or more. That is, it can be considered that GDL was hydrolyzed to generate gluconic acid, calcium carbonate released calcium ions, and sodium alginate was aggregated by leaving for 1 day at room temperature or more.

FIG. 7 shows a result of viscoelasticity measurement when the temperature of the dispersion was changed from 4° C. to 37° C. (assuming body temperature). It can be found that the storage elastic modulus (G′) and the loss elastic modulus (G″) of the dispersion crossed each other in about 30 minutes after the temperature was adjusted to 37° C., and the dispersion gelated (that is, sodium alginate aggregated).

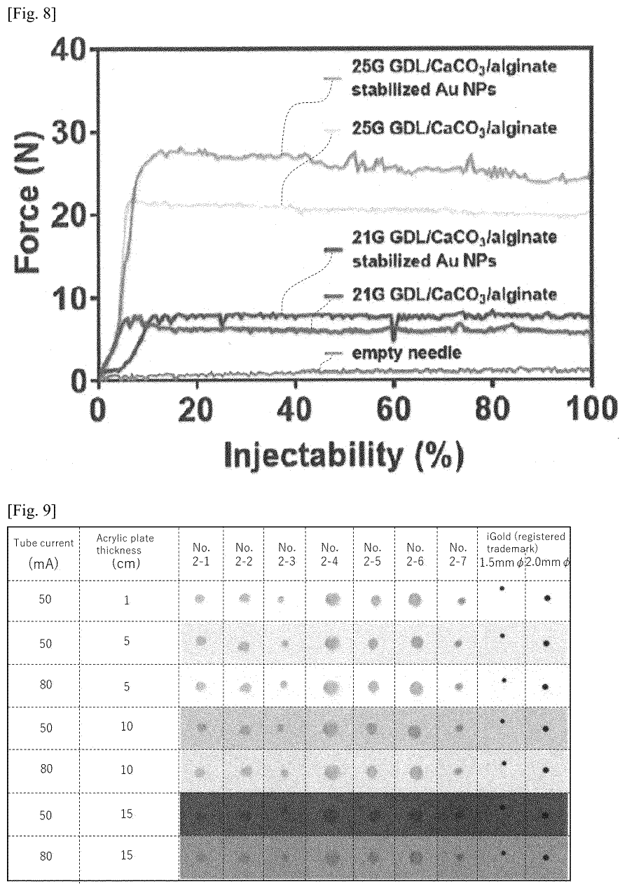

FIG. 8 shows a result of examining a force applied when the dispersion was injected with an injection needle (21 G (inner diameter: about 0.59 mm, needle length: about 38.1 mm), 25 G (inner diameter: about 0.25 mm, needle length: about 38.1 mm)). In FIG. 8, the horizontal axis represents the injection rate of the dispersion (Injectability, %), and the vertical axis represents the force (Force, N). In FIG. 8, “25 G GDL/CaCO₃/alginate stabilized Au NPs” was obtained by passing the dispersion through a 25 G injection needle, “25 G GDL/CaCO₃/alginate” was obtained by passing the dispersion containing no gold nanoparticles through a 25 G injection needle, “21 G GDL/CaCO₃/alginate stabilized Au NPs” was obtained by passing the dispersion through a 21 G injection needle, and “21 G GDL/CaCO₃/alginate” is obtained by passing the dispersion containing no gold nanoparticles through a 21 G injection needle. In FIG. 8, the mark “empty needle” indicates a force when the injection needle was pressed without putting the dispersion.

In FIG. 8, in the dispersion, injection was performed with a very weak force of 10 N or less with a 21 G injection needle, and injection was performed with a weak force of 30 N or less with a 25 G injection needle.

Using the dispersion for an X-ray target obtained as described above, aggregates (X-ray target) were produced in a simulated organ gel in the same manner as in Example 1, and X-ray visibilities were examined. The sizes of the X-ray targets were varied in 7 steps (Sample Nos. 2-1 to 2-7). The X-ray fluoroscopic images are shown in FIG. 9, and the gold contents of the X-ray targets and the visibility evaluation results (average value (%) of the correlation coefficient) are summarized in Table 3.

TABLE 3

| | Acrylic | Sample Nos. | | | | | | | iGold (registered trademark) | |
|---|---|---|---|---|---|---|---|---|---|---|
| Tube | plate | 2-1 | 2-2 | 2-3 | 2-4 | 2-5 | 2-6 | 2-7 | 1.5 mmφ | 2.0 mmφ |
| current | thickness | Gold | Gold | Gold | Gold | Gold | Gold | Gold | Gold | Gold |
| (mA) | (cm) | 1.82 mg | 3.52 mg | 3.73 mg | 4.40 mg | 6.93 mg | 8.92 mg | 9.84 mg | 34.2 mg | 80.95 mg |
| 50 | 1 | 88.35 | 91.20 | 92.94 | 93.01 | 94.27 | 94.65 | 96.16 | 99.00 | 99.53 |
| 50 | 5 | 70.71 | 72.70 | 75.84 | 78.59 | 82.22 | 83.26 | 87.52 | 97.38 | 98.52 |
| 80 | 5 | 80.84 | 82.93 | 86.12 | 86.88 | 89.41 | 89.73 | 92.39 | 98.04 | 99.14 |
| 50 | 10 | 33.98 | 39.96 | 40.90 | 43.68 | 50.43 | 53.29 | 62.74 | 90.04 | 94.45 |
| 80 | 10 | 43.63 | 52.32 | 56.44 | 59.60 | 63.52 | 65.23 | 72.46 | 93.63 | 96.46 |
| 50 | 15 | 1.00 | 1.00 | 1.23 | 1.00 | 1.00 | 1.84 | 16.10 | 67.60 | 76.97 |
| 80 | 15 | 1.67 | 1.65 | 10.41 | 10.02 | 25.17 | 26.83 | 40.25 | 77.37 | 87.40 |

As can be seen from Table 3, in Sample Nos. 2-1 to 2-7, the gold contents were 1.5 mg or more, and sufficient X-ray visibilities (specifically, the average value of correlation coefficients exceeds 30% at a tube current of 50 mA and an acrylic plate of 10 cm) were obtained. It can be considered that X-ray visibilities became better as the gold content was increased, and X-ray visibility (average value (%) of correlation coefficient) substantially equivalent to that of iGold (registered trademark) could be ensured by setting the gold content to 22.0 mg or more as in Example 1.

### Example 3

Chloroauric acid was reduced by an alcohol reduction method in the presence of a calcium phosphate-based bone reinforcing material as an aggregatable material to obtain a dispersion in which gold nanoparticles were in contact with the calcium phosphate-based bone reinforcing material.

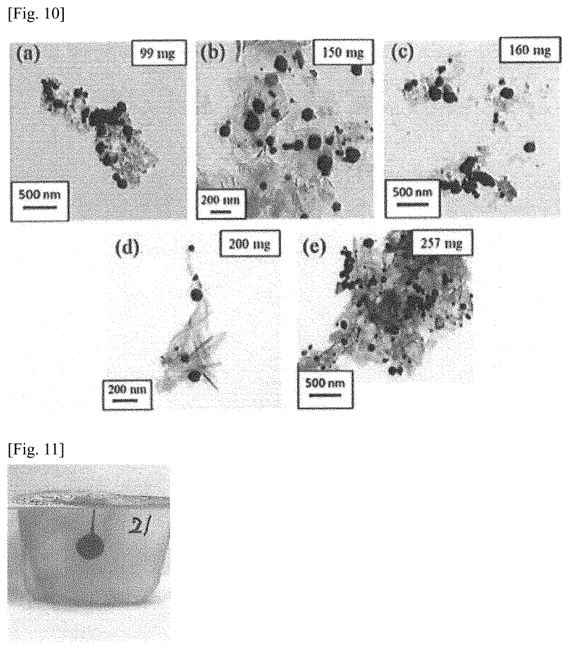

Specifically, a mixed solution of pure water:ethanol in a volume ratio of 1:1 and BIOPEX (registered trademark)-R (powder for excellent type 9 mL) (Average particle size (median diameter): 3.819 um, manufactured by HOYA Technosurgical Co., Ltd.) were put in a 2 L two-necked flask, and heated and boiled in an oil bath. A 24.6 mM chloroauric acid aqueous solution was then added. The final chloroauric acid concentration was 0.50 to 1.00 mM. The addition amount of BIOPEX (registered trademark)-R was 99, 150, 160, 200 or 257 mg. The stirring speed was 700 rpm, and the heating time was 2 to 6 hours. The dispersion after the reaction was dispersed by an ultrasonic cleaner for 10 minutes, and then centrifuged for 10 minutes to be washed and collected. The collected powder in which the gold nanoparticles were in contact with BIOPEX (registered trademark)-R after collection was vacuum-dried for 5 hours. FIG. 10 shows results of TEM observation of powders obtained when the addition amount of BIOPEX (registered trademark)-R was set to 99 mg (a), 150 mg (b), 160 mg (c), 200 mg (d), and 257 mg (e). In FIG. 10, it can be seen that the gold nanoparticles indicated by the dark color portion were in contact with the calcium phosphate-based bone reinforcing material indicated by a light color portion. From FIG. 10, it can be considered that the average particle size (median diameter) of the gold nanoparticles was 100 nm or less at most.

The powder for an X-ray target (one in FIG. 10(e)) obtained as described above was dispersed in water, and a dedicated kneading liquid (manufactured by HOYA Technosurgical) of BIOPEX (registered trademark)-R as a reaction promoting liquid was added thereto at a ratio of 1 mL to 3 g of BIOPEX (registered trademark)-R. Using the obtained dispersion for an X-ray target (gold nanoparticles concentration: 1.25 mg/mm³), an aggregation experiment in a simulated organ gel was performed. FIG. 11 shows a photograph after injecting the dispersion into two places in the simulated organ gel (KONNYAKUBATAKE, manufactured by MannanLife CO., LTD.) with an injection needle 21 G (inner diameter: about 0.59 mm, needle length: about 38.1 mm). As shown in FIG. 11, by using the dispersion, a 21 G injection needle could be passed with a weak force, and an aggregate (dark color part) could be formed in the simulated organ gel.

Using the dispersion for an X-ray target obtained as described above, aggregates (X-ray target) were produced in a simulated organ gel so that the gold content was at least 1.5 mg or more (Sample No. 3-1). The X-ray visibility was examined in the same manner as in Example 1. Table 4 shows a summary of the evaluation results (average values (%) of correlation coefficients).

TABLE 4

| Tube | Acrylic plate | Sample Nos. | iGold (registered trademark) | |
|---|---|---|---|---|
| current | thickness | 3-1 | 1.5 mmφ | 2.0 mmφ |
| (mA) | (cm) | Gold ≥ 1.5 mg | Gold 34.2 mg | Gold 80.95 mg |
| 50 | 1 | 100.00 | 99.01 | 99.56 |
| 50 | 5 | 98.77 | 97.08 | 98.58 |
| 80 | 5 | 100.00 | 98.39 | 99.15 |
| 50 | 10 | 86.37 | 88.25 | 94.75 |
| 80 | 10 | 93.68 | 93.72 | 96.75 |
| 50 | 15 | 47.24 | 61.43 | 80.17 |
| 80 | 15 | 68.67 | 80.24 | 88.19 |
| 50 | 20 | 1.00 | 12.00 | 37.39 |
| 80 | 20 | 2.28 | 36.74 | 58.43 |
| 160 | 20 | 37.40 | 51.61 | 77.88 |
| 80 | 25 | 1.00 | 1.00 | 9.35 |
| 160 | 25 | 1.00 | 3.84 | 24.70 |

As can be seen from Table 4, in Sample No. 3-1, the gold content was 1.5 mg or more, and sufficient X-ray visibility (specifically, the average value of correlation coefficients exceeds 30% at a tube current of 50 mA and an acrylic plate of 10 cm) was ensured. It can be considered that X-ray visibility (average value (%) of correlation coefficient) substantially equivalent to that of iGold (registered trademark) can be ensured by setting the gold content to 22.0 mg or more.

### Example 4

A gold rod (that is, a rod made of gold) was reduced by an in-liquid plasma method in the presence of sodium alginate as an aggregatable material to obtain a dispersion of gold nanoparticles whose surface was covered with sodium alginate. Details thereof will be described below.

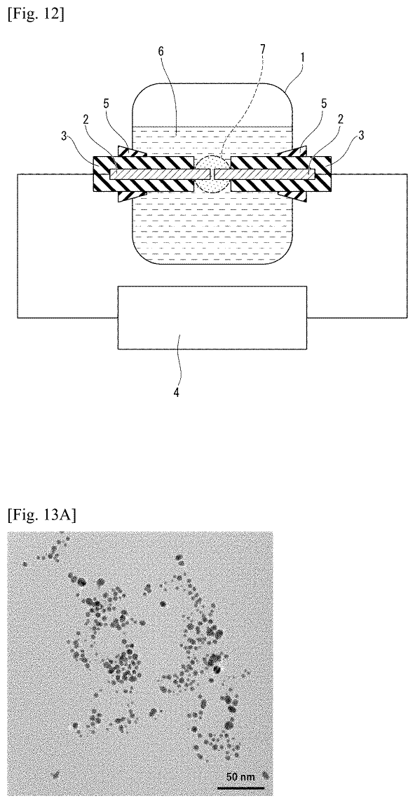

FIG. 12 is a schematic view for explaining an in-liquid plasma method performed in Example 4. As shown in FIG. 12, two 3 mmφ gold rods 2 were placed in a sealable flask 1, and these gold rods 2 were inserted into ceramic tubes 3, respectively, and connected to a radio wave generator 4. The distance between the two gold rods 2 was 1 mm. A silicone stoppers 5 were disposed at the insertion port of the ceramic tubes 3 in the flask 1, respectively, to ensure the sealability in the flask 1. A 0.002% aqueous sodium alginate solution (weight/volume (g/ml) ratio) 6 was put into the flask 1, and the gold rods 2 were immersed in the aqueous solution 6. A pressure in the flask 1 was set to 250 hPa, a power was set to 100 to 150 W, and plasma 7 was generated between the two gold rods 2 to obtain a dispersion of gold nanoparticles whose surface was covered with sodium alginate.

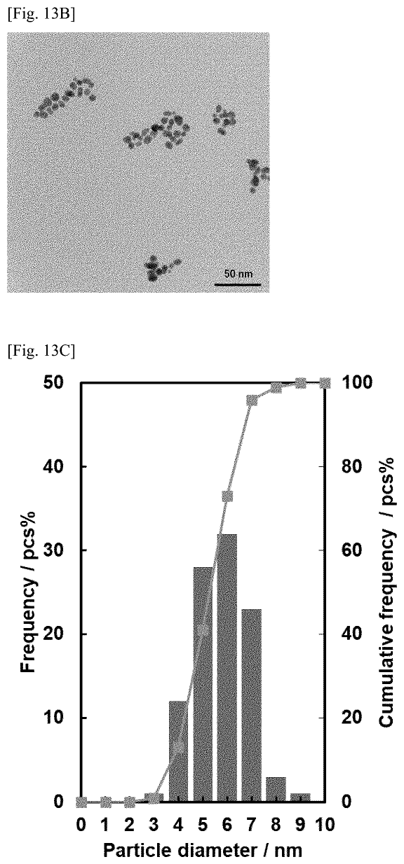

The gold nanoparticle dispersion obtained as described above was centrifuged at 0° C., at 12000 rpm and for 24 hours. The particle size distribution of the gold nanoparticles before and after centrifugation was determined by observing the particle size distribution with TEM. FIG. 13A shows an example of a TEM image of gold nanoparticles before centrifugation, and FIG. 13B shows an example of a TEM image of gold nanoparticles after centrifugation. As can be seen from FIGS. 13A and 13B, the gold nanoparticles were more separated by centrifugation. FIG. 13C shows a particle size distribution of gold nanoparticles determined from an optional region of a TEM image after centrifugation. The horizontal axis in FIG. 13C represents the particle size (nm) of the gold nanoparticles. For example, when the particle size is “5 nm”, this means “5.0 nm or more and less than 6.0 nm”. The bar graph in FIG. 13C indicates the frequency (%) (refer to the left vertical axis) at a predetermined particle size, and the line graph indicates the cumulative frequency (%) (refer to the right vertical axis). The gold nanoparticles of Example 4 had an average particle size (median diameter) of 6.0 nm, which was very small as compared with the gold nanoparticles of Example 1. From this result, it can be considered that the dispersion for an X-ray target using the gold nanoparticles of Example 4 can be injected into the body with an injection needle having a diameter smaller than that of Example 1 (that is, 21 G or more).

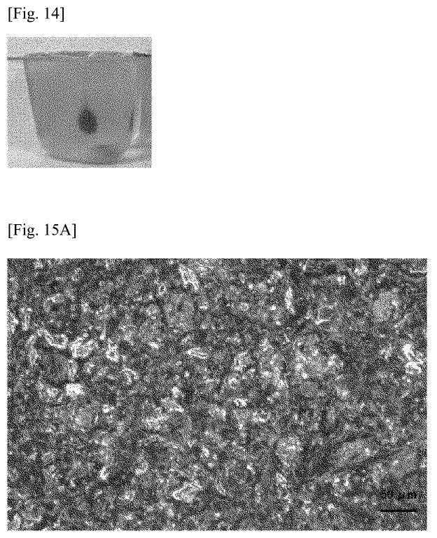

The gold nanoparticles whose surfaces were covered with sodium alginate and obtained as described above after centrifugation were redispersed in an aqueous sodium alginate solution to prepare a dispersion for an X-ray target (gold nanoparticle concentration: 1.0 mg/mm³, sodium alginate concentration: 5.0% (mass/volume (g/ml) ratio)). Using the dispersion, an aggregation experiment in a simulated organ gel was performed. FIG. 14 shows a photograph after injecting the dispersion into a simulated organ gel (KONNYAKUBATAKE manufactured by manufactured by MannanLife CO., LTD.) with an injection needle, and subsequently injecting an aqueous solution in which calcium chloride (1.8 mM) and magnesium chloride hexahydrate (1.5 mM) were dissolved. As shown in FIG. 14, by using the dispersion, aggregates (dark color portions) could be formed in the simulated organ gel.

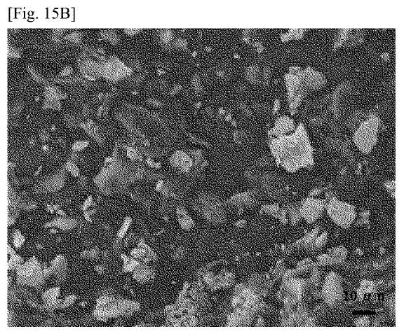

FIG. 15A shows a surface image of the X-ray target aggregated in the simulated organ gel as described above, which was photographed with a digital microscope. As can be seen in FIG. 15A, the X-ray target was confirmed to have gold grains of various sizes spread relatively uniformly. FIG. 15B shows a surface SEM image of the X-ray target. In FIG. 15B, the gold grains are observed as white. In FIG. 15B, the gold grains were shown without being particularly biased.

Using the dispersion for an X-ray target obtained as described above, an aggregate (X-ray target) was produced in a simulated organ gel so that the gold content was at least 1.5 mg or more (Sample No. 4-1). Then, the X-ray visibility was examined in the same manner as in Example 1. Table 5 shows a summary of the evaluation results (average values (%) of correlation coefficients).

TABLE 5

| Tube | Acrylic plate | Sample Nos. | iGold (registered trademark) | |
|---|---|---|---|---|
| current | thickness | 4-1 | 1.5 mmφ | 2.0 mmφ |
| (mA) | (cm) | Gold ≥ 1.5 mg | Gold 34.2 mg | Gold 80.95 mg |
| 50 | 1 | 98.75 | 99.12 | 99.50 |
| 50 | 5 | 95.93 | 97.38 | 98.52 |
| 80 | 5 | 97.76 | 98.73 | 99.27 |
| 50 | 10 | 82.32 | 92.01 | 94.20 |
| 80 | 10 | 90.05 | 95.19 | 97.35 |
| 50 | 15 | 51.35 | 61.90 | 77.86 |
| 80 | 15 | 67.02 | 78.51 | 87.82 |
| 50 | 20 | 0 | 20.60 | 37.91 |
| 80 | 20 | 26.89 | 42.96 | 54.98 |
| 160 | 20 | 35.71 | 57.84 | 75.92 |
| 80 | 25 | 0 | 0 | 0 |
| 160 | 25 | 0 | 16.00 | 11.76 |

As can be seen from Table 5, in Sample No. 4-1, the gold content was 1.5 mg or more, and sufficient X-ray visibility (specifically, the average value of correlation coefficients exceeds 30% at a tube current of 50 mA and an acrylic plate of 10 cm) was ensured. It can be considered that X-ray visibility (average value (%) of correlation coefficient) substantially equivalent to that of iGold (registered trademark) can be ensured by setting the gold content to 22.0 mg or more.

This application claims priority based on Japanese Application No. 2021-029450 filed on Feb. 26, 2021, the disclosure of which is incorporated by reference herein.

1. A dispersion for an X-ray target wherein gold nanoparticles and sodium alginate or a calcium phosphate-based bone reinforcing material are dispersed,
wherein the gold nanoparticles are in contact with the sodium alginate or the calcium phosphate-based bone reinforcing material.

2. A prefilled syringe filled with the dispersion according to claim 1, wherein the gold nanoparticles and the sodium alginate are dispersed and a calcium ion solution in a non-mixed state.

3. The dispersion according to claim 1, wherein the gold nanoparticles and the sodium alginate are dispersed, and calcium carbonate and lactones are further contained.

4. A prefilled syringe filled with the dispersion according to claim 3.

5. A powder for an X-ray target comprising:
a gold nanoparticle; and
a calcium phosphate-based bone reinforcing material,
wherein the gold nanoparticle is in contact with the calcium phosphate-based bone reinforcing material.
